(19) United States
(12) Patent Application Publication
(10) Pub. No.: US 2015/0284393 A1
(43) Pub. Date: Oct. 8, 2015

(54) METHODS FOR TREATING EYE DISORDERS

(71) Applicant: PRISM PHARMA CO., LTD, Kanagawa (JP)

(72) Inventors: Hiroyuki Kouji, Kanagawa (JP); Takenao Odagami, Kanagawa (JP)

(73) Assignee: PRISM Pharma Co., Ltd., Kanagawa (JP)

(21) Appl. No.: 14/436,689

(22) PCT Filed: Oct. 21, 2013
(86) PCT No.: PCT/JP2013/079053

§ 371 Date: Apr. 17, 2015

Related U.S. Application Data
(60) Provisional application No. 61/716,186, filed on Oct. 19, 2012.

(51) Int. Cl. C07D 487/04

(52) U.S. Cl. CPC C07D 487/04

(57) ABSTRACT

The present disclosure relates generally to alpha-helix mimetic structures and specifically to alpha-helix mimetic structures that are inhibitors of β-catenin. The disclosure also relates to applications in the treatment of ophthalmic conditions, such as macular degeneration and glaucoma, and pharmaceutical compositions comprising such alpha helix mimetic β-catenin inhibitors.

### BACKGROUND OF THE DISCLOSURE

The Wnt gene family encodes a large class of secreted proteins related to the Int1/Wnt1 proto-oncogene and Drosophila wingless (“Wg”), a Drosophila Wnt1 homologue (Cadigan et al. (1997) Genes & Development 11:3286-3305). Wnts are expressed in a variety of tissues and organs and are required for many developmental processes, including segmentation in Drosophila; endoderm development in C. elegans; and establishment of limb polarity, neural crest differentiation, kidney morphogenesis, sex determination, and brain development in mammals (Parr, et al. (1994) Curr. Opinion Genetics & Devel. 4:523-528). The Wnt pathway is a master regulator in development, both during embryogenesis and in the mature organism (Eastman, et al. (1999) Curr Opin Cell Biol 11: 233-240; Peifer, et al. (2000) Science 287: 1606-1609).

Wnt signals are transduced by the Frizzled (“Fz”) family of seven transmembrane domain receptors (Bhanot et al. (1996) Nature 382:225-230). Frizzled cell-surface receptors (Fzd) play an essential role in both canonical and non-canonical Wnt signaling. In the canonical pathway, upon activation of Fzd and LRP5/6 (low-density-lipoprotein receptor-related protein 5 and 6) by Wnt proteins, a signal is generated that prevents the phosphorylation and degradation of β-catenin by the “β-catenin destruction complex,” permitting stable β-catenin translocation and accumulation in the nucleus, and therefore Wnt signal transduction. (Perrimon (1994) Cell 76:781-784)(Miller, J. R. (2001) Genome Biology; 3(1):1-15). The non-canonical Wnt signaling pathway is less well defined: there are at least two non-canonical Wnt signaling pathways that have been proposed, including the planar cell polarity (PCP) pathway, the Wnt/Ca++ pathway, and the convergence extension pathway.

Glycogen synthase kinase 3 (GSK3), the tumor suppressor gene product APC (adenomatous polyposis coli) (Gumbiner (1997) Curr. Biol. 7:R443-436), and the scaffolding protein Axin, are all negative regulators of the Wnt pathway, and together form the “β-catenin destruction complex.” In the absence of a Wnt ligand, these proteins form a complex and promote phosphorylation and degradation of β-catenin, whereas Wnt signaling inactivates the complex and prevents β-catenin degradation. Stabilized β-catenin translocates to the nucleus as a result, where it binds TCF (T cell factor) transcription factors (also known as lymphoid enhancer-binding factor-1 (LEF1)) and serves as a coactivator of TCF/LEF-induced transcription (Bienz, et al. (2000) Cell 103: 311-320; Polakis, et al. (2000) Genes Dev 14: 1837-1851).

Wnt signaling occurs via canonical and non-canonical mechanisms. In the canonical pathway, upon activation of Fzd and LRP5/6 by Wnt proteins, stabilized β-catenin accumulates in the nucleus and leads to activation of TCF target genes (as described above; Miller, J. R. (2001) Genome Biology; 3(1):1-15). The non-canonical Wnt signaling pathway is less well defined: at least two non-canonical Wnt signaling pathways have been proposed, including the planar cell polarity (PCP) pathway and the Wnt/Ca++ pathway.

Diseases and degenerative conditions of the optic nerve and retina are the leading causes of blindness in the world. Macular degeneration (MD) is the loss of photoreceptors in the portion of the central retina, termed the macula, responsible for high-acuity vision. Age-related macular degeneration (AMD) is described as either “dry” or “wet.” The wet, exudative, neovascular form of AMD affects about 10% of those with AMD and is characterized by abnormal blood vessels growing through the retinal pigment epithelium (RPE), resulting in hemorrhage, exudation, scarring, or serous retinal detachment. Ninety percent of AMD patients have the dry form characterized by atrophy of the retinal pigment epithelium and loss of macular photoreceptors. At present there is no cure for any form of MD or AMD, although some success in attenuation has been obtained with photodynamic therapy.

Glaucoma is a condition resulting from several distinct eye diseases that cause vision loss by damage to the optic nerve. Elevated intraocular pressure (IOP) due to inadequate ocular drainage is the most frequent cause of glaucoma. Glaucoma often develops as the eye ages, or it can occur as the result of an eye injury, inflammation, tumor or in advanced cases of cataract or diabetes. It can also be caused by the increase in IOP caused by treatment with steroids. Drug therapies that are proven to be effective in glaucoma reduce IOP either by decreasing vitreous humor production or by facilitating ocular draining. Such agents are often vasodilators and as such act on the sympathetic nervous system and include adrenergic antagonists.

There is an urgent need for new treatments for ophthalmic disorders such as macular degeneration (MD), age-related macular degeneration (AMD), glaucoma, cataracts, retinitis pigmentosa, choroidal neovascularization, retinal degeneration, and oxygen-induced retinopathy.

### BRIEF SUMMARY OF THE DISCLOSURE

The present disclosure relates generally to alpha-helix mimetic structures and specifically to alpha-helix mimetic structures that are inhibitors of β-catenin. The disclosure also relates to applications in the treatment of ophthalmic conditions, such as macular degeneration and glaucoma, and pharmaceutical compositions comprising such alpha helix mimetic β-catenin inhibitors.

### BRIEF DESCRIPTION OF THE FIGURES

FIGS. 1A-1D. Number of dividing immune cells, glial cells, astrocytes and Muller cells, following treatment with Compound A. Compound A is 4-(((6S,9S,9aS)-1-(benzylcarbamoyl)-2,9-dimethyl-4,7-dioxo-8-(quinolin-8-ylmethyl)octahydro-1H-pyrazino[2,1-c][1,2,4]triazin-6-yl)methyl)phenyl dihydrogen phosphate. (A), Compound A treatment did not affect immune cell response. (B-D), Increase in number of proliferating glial cells (A), Muller cells (B), and astrocytes (D) following retinal detachment is attenuated in eyes treated with Compound A relative to control vehicle levels.

FIGS. 2A-2B. Quantitative analysis of glial scar frequency and size following treatment with Compound A. (A), Frequency of glial scars is significantly reduced following treatment with Compound A. (B), Average glial scar length is significantly reduced following treatment with Compound A.

FIGS. 3A-3D. Immunohistochemistry identifies subretinal gliosis (or scarring) seen as the presence of vimentin labeled Muller cell processes extending into the subretinal space. OS, outer layer/subretinal space; ONL, outer nuclear layer; GCL, ganglion cell layer. (A-B), Vehicle treated eyes. Following detachment, vimentin expression increases in Muller cells, and Muller cell processes are seen extending into the subretinal space (arrows). Arrowheads point to dividing Muller cells. (C-D), Compound A treated eyes. No Muller cell growth into the subretinal space was observed. One dividing cell (astrocyte) is present in the GCL (arrow).

FIGS. 4A-4B. CNV lesion size following treatment with Compound A or Compound C. Compound C is (6S,9S,9aS)-N-benzyl-6-(4-hydroxybenzyl)-2,9-dimethyl-4,7-dioxo-8-(quinolin-8-ylmethyl)octahydro-1H-pyrazino[2,1-c][1,2,4]triazine-1-carboxamide. (A), Average CNV lesion size at day 15. (B), Average CNV lesion size at day 22.

### DETAILED DESCRIPTION OF THE DISCLOSURE

Recently, non-peptide compounds have been developed which mimic the secondary structure of reverse-turns found in biologically active proteins or peptides. For example, U.S. Pat. No. 5,440,013 and published PCT Applications Nos. WO94/03494, WO01/00210A1, and WO01/16135A2 each disclose conformationally constrained, non-peptidic compounds, which mimic the three-dimensional structure of reverse-turns. In addition, U.S. Pat. No. 5,929,237 and its continuation-in-part U.S. Pat. No. 6,013,458, disclose conformationally constrained compounds which mimic the secondary structure of reverse-turn regions of biologically active peptides and proteins. In relation to reverse-turn mimetics, conformationally constrained compounds have been disclosed which mimic the secondary structure of alpha-helix regions of biologically active peptide and proteins in WO2007/056513 and WO2007/056593.

The relevant structures and compounds of the alpha helix mimetic β-catenin inhibitors of this invention are disclosed in WO 2010/044485, WO 2010/128685, WO 2009/148192, and US 2011/0092459, each of which is incorporated herein by reference in its entirety. These compounds have now been found to be useful in the treatment of ophthalmic conditions and disorders, such as macular degeneration and glaucoma. While not wishing to be bound, the effectiveness of these compounds in treating these conditions is based in part on the ability of these compounds to inhibit β-catenin, thus altering Wnt pathway signaling, which has been found to improve various ophthalmic diseases and conditions.

The preferable structure of the alpha helix mimetic β-catenin inhibitors of this invention have the following formula (I):

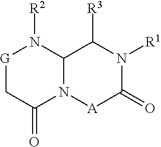

wherein
A is —CHR⁷—,
   wherein
   R⁷ is optionally substituted arylalkyl, optionally substituted heteroarylalkyl, optionally substituted cycloalkylalkyl or optionally substituted heterocycloalkylalkyl;
G is —NH—, —NR⁶—, or —O—
   wherein
   R⁶ is lower alkyl or lower alkenyl;
R¹ is —Ra—R¹⁰;
   wherein
   Ra is optionally substituted lower alkylene and
   R¹⁰ is optionally substituted bicyclic fused aryl or optionally substituted bicyclic fused heteroaryl;
R² is —(CO)—NH—Rb—R²⁰,
   wherein
   Rb is bond or optionally substituted lower alkylene; and
   R²⁰ is optionally substituted aryl or optionally substituted heteroaryl; and
R³ is C₁₋₄ alkyl.
   These compounds are especially useful in the prevention and/or treatment of ophthalmic conditions, such as macular degeneration and glaucoma.

The more preferable structure of the alpha helix mimetic β-catenin inhibitors of this invention have the following substituents in the above-mentioned formula (I):

### A is —CHR⁷—,

wherein
R⁷ is arylalkyl optionally substituted with hydroxyl or C₁₋₄ alkyl;

### G is —NH—, —NR⁶—, or —O—

wherein
R⁶ is C₁₋₄ alkyl or C₁₋₄ alkenyl;

### R¹ is —Ra—R¹⁰;

wherein
Ra is C₁₋₄ alkylene and
R¹⁰ is bicyclic fused aryl or bicyclic fused heteroaryl, optionally substituted with halogen or amino;

### R² is —(CO)—NH—Rb—R²⁰,

wherein
Rb is bond or C₁₋₄ alkylene; and
R²⁰ is aryl or heteroaryl; and
   R³ is C₁₋₄ alkyl.
   These compounds are especially useful in the prevention and/or treatment of ophthalmic conditions, such as macular degeneration and glaucoma.

The most preferable alpha helix mimetic β-catenin inhibitors of this invention are as follows:

(6S,9S)—N-benzyl-6-(4-hydroxybenzyl)-2,9-dimethyl-8-(naphthalen-1-ylmethyl)-4,7-dioxooctahydro-1H-pyrazino[2,1-c][1,2,4]triazine-1-carboxamide,

(6S,9S)-2-allyl-N-benzyl-6-(4-hydroxybenzyl)-9-methyl-8-(naphthalen-1-ylmethyl)-4,7-dioxooctahydro-1H-pyrazino[2,1-c][1,2,4]triazine-1-carboxamide,

(6S,9S)—N-benzyl-6-(4-hydroxybenzyl)-9-methyl-8-(naphthalen-1-ylmethyl)-4,7-dioxohexahydropyrazino[2,1-c][1,2,4]oxadiazine-1(6H)-carboxamide,

(6S,9S)-8-((2-aminobenzo[d]thiazol-4-yl)methyl)-N-benzyl-6-(4-hydroxybenzyl)-2,9-dimethyl-4,7-dioxooctahydro-1H-pyrazino[2,1-c][1,2,4]triazine-1-carboxamide,

(6S,9S)—N-benzyl-6-(4-hydroxybenzyl)-2,9-dimethyl-4,7-dioxo-8-(quinolin-8-ylmethy)octahydro-1H-pyrazino[2,1-c][1,2,4]triazine-1-carboxamide,

(6S,9S)-2-allyl-N-benzyl-6-(4-hydroxybenzyl)-9-methyl-4,7-dioxo-8-(quinolin-8-ylmethyl)octahydro-1H-pyrazino[2,1-c][1,2,4]triazine-1-carboxamide,

4-(((6S,9S)-1-(benzylcarbamoyl)-2,9-dimethyl-4,7-dioxo-8-(quinolin-8-ylmethyl)octahydro-1H-pyrazino[2,1-c][1,2,4]triazin-6-yl)methyl)phenyl dihydrogen phosphate,

4-(((6S,9S)-1-(benzylcarbamoyl)-2,9-dimethyl-8-(naphthalen-1-ylmethyl)-4,7-dioxooctahydro-1H-pyrazino[2,1-c][1,2,4]triazin-6-yl)methyl)phenyl dihydrogen phosphate,

sodium 4-(((6S,9S)-1-(benzylcarbamoyl)-2,9-dimethyl-4,7-dioxo-8-(quinolin-8-ylmethyl)octahydro-1H-pyrazino[2,1-c][1,2,4]triazin-6-yl)methyl)phenyl phosphate,

sodium 4-(((6S,9S)-1-(benzylcarbamoyl)-2,9-dimethyl-4,7-dioxo-8-(naphthalen-8-ylmethyl)octahydro-1H-pyrazino[2,1-c][1,2,4]triazin-6-yl)methyl)phenyl phosphate,

(6S,9S)-2-allyl-6-(4-hydroxybenzyl)-9-methyl-4,7-dioxo-N—((R)-1-phenylethyl)-8-(quinolin-8-ylmethyl)octahydro-1H-pyrazino[2,1-c][1,2,4]triazine-1-carboxamide,

(6S,9S)-2-allyl-6-(4-hydroxybenzyl)-9-methyl-4,7-dioxo-N—((S)-1-phenylethyl)-8-(quinolin-8-ylmethyl)octahydro-1H-pyrazino[2,1-c][1,2,4]triazine-1-carboxamide,

(6S,9S)—N-benzyl-6-(4-hydroxy-2,6-dimethylbenzyl)-2,9-dimethyl-4,7-dioxo-8-(quinolin-8-ylmethyl)octahydro-1H-pyrazino[2,1-c][1,2,4]triazine-1-carboxamide,

(6S,9S)-8-(benzo[b]thiophen-3-ylmethyl)-N-benzyl-6-(4-hydroxybenzyl)-2,9-dimethyl-4,7-dioxooctahydro-1H-pyrazino[2,1-c][1,2,4]triazine-1-carboxamide,

(6S,9S)-8-(benzo[c][1,2,5]thiadiazol-4-ylmethyl)-N-benzyl-6-(4-hydroxybenzyl)-2,9-dimethyl-4,7-dioxooctahydro-1H-pyrazino[2,1-c][1,2,4]triazine-1-carboxamide,

(6S,9S)—N-benzyl-6-(4-hydroxybenzyl)-8-(isoquinolin-5-ylmethyl)-2,9-dimethyl-4,7-dioxooctahydro-1H-pyrazino[2,1-c][1,2,4]triazine-1-carboxamide,

(6S,9S)—N-benzyl-8-((5-chlorothieno[3,2-b]pyridin-3-yl)methyl)-6-(4-hydroxybenzyl)-2,9-dimethyl-4,7-dioxooctahydro-1H-pyrazino[2,1-c][1,2,4]triazine-1-carboxamide,

(6S,9 S)—N-benzyl-6-(4-hydroxybenzyl)-2,9-dimethyl-4,7-dioxo-8-(quinoxalin-5-ylmethyl)octahydro-1H-pyrazino[2,1-c][1,2,4]triazine-1-carboxamide, and

(6S,9S)-6-(4-hydroxybenzyl)-2,9-dimethyl-4,7-dioxo-8-(quinolin-8-ylmethyl)-N-(thiophen-2-ylmethyl)octahydro-1H-pyrazino[2,1-c][1,2,4]triazine-1-carboxamide.

These compounds are especially useful in the prevention and/or treatment of ophthalmic conditions, such as macular degeneration and glaucoma.

In a most preferred embodiment, the compound is:

4-(((6S,9S,9aS)-1-(benzylcarbamoyl)-2,9-dimethyl-4,7-dioxo-8-(quinolin-8-ylmethyl)octahydro-1H-pyrazino[2,1-c][1,2,4]triazin-6-yl)methyl)phenyl dihydrogen phosphate (Compound A), or

(6S,9S,9aS)-N-benzyl-6-(4-hydroxybenzyl)-2,9-dimethyl-4,7-dioxo-8-(quinolin-8-ylmethyl)octahydro-1H-pyrazino[2,1-c][1,2,4]triazine-1-carboxamide (Compound C).

These compounds are especially useful in the prevention and/or treatment of ophthalmic conditions, such as macular degeneration and glaucoma.

In particular, the alpha helix mimetics of the invention have been found to be useful as inhibitors of β-catenin. Disclosed herein are alpha helix mimetic β-catenin inhibitor compounds for treatment of ophthalmic diseases and conditions.

A “β-catenin inhibitor” is a substance that can reduce or prevent β-catenin activity. β-catenin activities include translocation to the nucleus, binding with TCF (T cell factor) transcription factors, and coactivating TCF transcription factor-induced transcription of TCF target genes.

An “ophthalmic disease” or “ophthalmic condition” can be any disease, condition or disorder that affects the eye and eye area, including but not limited to macular degeneration (MD), age-related macular degeneration (AMD), glaucoma, cataracts, retinitis pigmentosa, choroidal neovascularization, retinal degeneration, and oxygen-induced retinopathy.

As used herein, “treatment” refers to clinical intervention in an attempt to alter the disease course of the individual or cell being treated, and can be performed during the course of clinical pathology. Therapeutic effects of treatment include without limitation, preventing recurrence of disease, alleviation of symptoms, diminishment of any direct or indirect pathological consequences of the disease, decreasing the rate of disease progression, amelioration or palliation of the disease state, and remission or improved prognosis.

As used herein, the terms “therapeutically effective amount” and “effective amount” are used interchangeably to refer to an amount of a composition of the invention that is sufficient to result in the prevention of the development or onset of an ophthalmic disease, or one or more symptoms thereof, to enhance or improve the effect(s) of another therapy, and/or to ameliorate one or more symptoms of an ophthalmic disease.

A therapeutically effective amount can be administered to a patient in one or more doses sufficient to palliate, ameliorate, stabilize, reverse or slow the progression of the disease, or otherwise reduce the pathological consequences of the disease, or reduce the symptoms of the disease. The amelioration or reduction need not be permanent, but may be for a period of time ranging from at least one hour, at least one day, or at least one week or more. The effective amount is generally determined by the physician on a case-by-case basis and is within the skill of one in the art. Several factors are typically taken into account when determining an appropriate dosage to achieve an effective amount. These factors include age, sex and weight of the patient, the condition being treated, the severity of the condition, as well as the route of administration, dosage form and regimen and the desired result.

As used herein, the terms “subject” and “patient” are used interchangeably and refer to an animal, preferably a mammal such as a non-primate (e.g., cows, pigs, horses, cats, dogs, rats etc.) and a primate (e.g., monkey and human), and most preferably a human.

The alpha helix mimetic β-catenin inhibitors described herein can be incorporated into pharmaceutical compositions for administration, singly or in combination, to a subject for the treatment or prevention of a disorder described herein. Such compositions typically include the active agent and a pharmaceutically acceptable carrier. As used herein the term “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

The compounds and compositions described herein are useful for treatment of ophthalmic conditions and diseases, such as macular degeneration and glaucoma.

The alpha helix mimetic β-catenin inhibitors described herein are useful to prevent or treat disease. Specifically, the disclosure provides for both prophylactic and therapeutic methods of treating a subject at risk of (or susceptible to) an ophthalmic disease or condition. Accordingly, the present methods provide for the prevention and/or treatment of an ophthalmic condition in a subject by administering an effective amount of an alpha helix mimetic β-catenin inhibitor to a subject in need thereof. For example, a subject can be administered a β-catenin inhibitor composition in an effort to improve one or more of the factors contributing to an ophthalmic disease or condition.

One aspect of the technology includes methods of reducing an ophthalmic condition in a subject for therapeutic purposes. In therapeutic applications, compositions or medicaments are administered to a subject suspected of, or already suffering from such a disease in an amount sufficient to cure, or at least partially arrest, the symptoms of the disease, including its complications and intermediate pathological phenotypes in development of the disease. As such, the disclosure provides methods of treating an individual afflicted with an ophthalmic condition. In some embodiments, the technology provides a method of treating or preventing specific ophthalmic disorders, such as cataracts, retinitis pigmentosa, glaucoma, choroidal neovascularization, retinal degeneration, and oxygen-induced retinopathy, in a mammal by administering an alpha helix mimetic β-catenin inhibitor.

In one embodiment, the β-catenin inhibitor is administered to a subject to treat or prevent cataracts. Cataracts is a congenital or acquired disease characterized by a reduction in natural lens clarity. Individuals with cataracts may exhibit one or more symptoms, including, but not limited to, cloudiness on the surface of the lens, cloudiness on the inside of the lens, and/or swelling of the lens. Typical examples of congenital cataract-associated diseases are pseudo-cataracts, membrane cataracts, coronary cataracts, lamellar cataracts, punctuate cataracts, and filamentary cataracts. Typical examples of acquired cataract-associated diseases are geriatric cataracts, secondary cataracts, browning cataracts, complicated cataracts, diabetic cataracts, and traumatic cataracts. Acquired cataracts is also inducible by electric shock, radiation, ultrasound, drugs, systemic diseases, and nutritional disorders. Acquired cataracts further includes postoperative cataracts.

In one embodiment, the β-catenin inhibitor is administered to a subject to treat or prevent retinitis pigmentosa. Retinitis pigmentosa is a disorder that is characterized by rod and/or cone cell damage. The presence of dark lines in the retina is typical in individuals suffering from retinitis pigmentosa. Individuals with retinitis pigmentosa also present with a variety of symptoms including, but not limited to, headaches, numbness or tingling in the extremities, light flashes, and/or visual changes. See, e.g., Heckenlively et al., Am J. Ophthalmol. 105(5): 504-511 (1988).

In one embodiment, the β-catenin inhibitor is administered to a subject to treat or prevent glaucoma. Glaucoma is a genetic disease characterized by an increase in intraocular pressure, which leads to a decrease in vision. Glaucoma may emanate from various ophthalmologic conditions that are already present in an individual, such as, wounds, surgery, and other structural malformations. Although glaucoma can occur at any age, it frequently develops in elderly individuals and leads to blindness. Glaucoma patients typically have an intraocular pressure in excess of 21 mmHg. However, normal tension glaucoma, where glaucomatous alterations are found in the visual field and optic papilla, can occur in the absence of such increased intraocular pressures, i.e., greater than 21 mmHg. Symptoms of glaucoma include, but are not limited to, blurred vision, severe eye pain, headache, seeing haloes around lights, nausea, and/or vomiting.

In one embodiment, the β-catenin inhibitor is administered to a subject to treat or prevent macular degeneration. Macular degeneration is typically an age-related disease. The general categories of macular degeneration include wet, dry, and non-aged related macular degeneration. Dry macular degeneration, which accounts for about 80-90 percent of all cases, is also known as atrophic, nonexudative, or drusenoid macular degeneration. With dry macular degeneration, drusen typically accumulate beneath the retinal pigment epithelium tissue. Vision loss subsequently occurs when drusen interfere with the function of photoreceptors in the macula. Symptoms of dry macular generation include, but are not limited to, distorted vision, center-vision distortion, light or dark distortion, and/or changes in color perception. Dry macular degeneration can result in the gradual loss of vision.

Wet macular degeneration is also known as neovascularization, subretinal neovascularization, exudative, or disciform degeneration. With wet macular degeneration, abnormal blood vessels grow beneath the macula. The blood vessels leak fluid into the macula and damage photoreceptor cells. Wet macular degeneration can progress rapidly and cause severe damage to central vision. Wet and dry macular degeneration have identical symptoms. Non-age related macular degeneration, however, is rare and may be linked to heredity, diabetes, nutritional deficits, injury, infection, or other factors. The symptoms of non-age related macular degeneration also include, but are not limited to, distorted vision, center-vision distortion, light or dark distortion, and/or changes in color perception.

In one embodiment, the β-catenin inhibitor is administered to a subject to treat or prevent choroidal neovascularization. Choroidal neovascularization (CNV) is a disease characterized by the development of new blood vessels in the choroid layer of the eye. The newly formed blood vessels grow in the choroid, through the Bruch membrane, and invade the subretinal space. CNV can lead to the impairment of sight or complete loss of vision. Symptoms of CNV include, but are not limited to, seeing flickering, blinking lights, or gray spots in the affected eye or eyes, blurred vision, distorted vision, and/or loss of vision.

In one embodiment, the β-catenin inhibitor is administered to a subject to treat or prevent retinal degeneration. Retinal degeneration is a genetic disease that relates to the break-down of the retina. Retinal tissue may degenerate for various reasons, such as, artery or vein occlusion, diabetic retinopathy, retinopathy of prematurity, and/or retrolental fibroplasia. Retinal degradation generally includes retinoschisis, lattic degeneration, and is related to progressive macular degeneration. The symptoms of retina degradation include, but are not limited to, impaired vision, loss of vision, night blindness, tunnel vision, loss of peripheral vision, retinal detachment, and/or light sensitivity.

In one embodiment, the β-catenin inhibitor is administered to a subject to treat or prevent oxygen-induced retinopathy. Oxygen-induced retinopathy (OIR) is a disease characterized by microvascular degeneration. OIR is an established model for studying retinopathy of prematurity. OIR is associated with vascular cell damage that culminates in abnormal neovascularization. Microvascular degeneration leads to ischemia which contributes to the physical changes associated with OIR. Oxidative stress also plays an important role in the vasoobliteration of OIR where endothelial cells are prone to peroxidative damage. Pericytes, smooth muscle cells, and perivascular astrocytes, however, are generally resistant to peroxidative injury. See, e.g., Beauchamp et al., Role of thromboxane in retinal microvascular degeneration in oxygen-induced retinopathy, J Appl Physiol. 90: 2279-2288 (2001). OIR, including retinopathy of prematurity, is generally asymptomatic. However, abnormal eye movements, crossed eyes, severe nearsightedness, and/or leukocoria, can be a sign of OIR or retinopathy of prematurity.

In one aspect, the invention provides a method for preventing, in a subject, an ophthalmic condition by administering to the subject an alpha-helix mimetic β-catenin inhibitor that modulates one or more signs or markers of an ophthalmic condition. Subjects at risk for an ophthalmic condition can be identified by, e.g., any or a combination of diagnostic or prognostic assays. In prophylactic applications, pharmaceutical compositions or medicaments of the alpha helix mimetic β-catenin inhibitors are administered to a subject susceptible to, or otherwise at risk of a disease or condition in an amount sufficient to eliminate or reduce the risk, lessen the severity, or delay the outset of the disease, including biochemical, histologic and/or behavioral symptoms of the disease, its complications and intermediate pathological phenotypes presenting during development of the disease. Administration of the β-catenin inhibitors can occur prior to the manifestation of symptoms characteristic of the aberrancy, such that a disease or disorder is prevented or, alternatively, delayed in its progression.

Any suitable route of administration may be employed for providing a mammal, especially a human, with an effective dose of a compound described herein. For example, oral, rectal, topical, parenteral, ocular, pulmonary, nasal, and the like may be employed. Dosage forms include tablets, troches, dispersions, suspensions, solutions, capsules, creams, ointments, aerosols, and the like. Preferably compounds described herein are administered orally.

The effective dosage of active ingredient employed may vary depending on the particular compound employed, the mode of administration, the condition being treated and the severity of the condition being treated. Such dosage may be ascertained readily by a person skilled in the art.

When treating or controlling ophthalmic conditions and diseases for which compounds described herein are indicated, generally satisfactory results are obtained when the compounds described herein are administered at a daily dosage of from about 0.1 milligram to about 100 milligram per kilogram of animal body weight, preferably given as a single daily dose or in divided doses two to six times a day, or in sustained release form. For most large mammals, the total daily dosage is from about 1.0 milligrams to about 1000 milligrams. In the case of a 70 kg adult human, the total daily dose will generally be from about 1 milligram to about 500 milligrams. For a particularly potent compound, the dosage for an adult human may be as low as 0.1 mg. In some cases, the daily dose may be as high as 1 gram. The dosage regimen may be adjusted within this range or even outside of this range to provide the optimal therapeutic response.

Oral administration will usually be carried out using tablets or capsules. Examples of doses in tablets and capsules are 0.1 mg, 0.25 mg, 0.5 mg, 1 mg, 2 mg, 5 mg, 10 mg, 15 mg, 20 mg, 25 mg, 30 mg, 40 mg, 50 mg, 100 mg, 200 mg, 250 mg, 300 mg, 400 mg, 500 mg, and 750 mg. Other oral forms may also have the same or similar dosages.

Also described herein are pharmaceutical compositions which comprise a compound described herein and a pharmaceutically acceptable carrier. The pharmaceutical compositions described herein comprise a compound described herein or a pharmaceutically acceptable salt as an active ingredient, as well as a pharmaceutically acceptable carrier and optionally other therapeutic ingredients. A pharmaceutical composition may also comprise a prodrug, or a pharmaceutically acceptable salt thereof, if a prodrug is administered.

The compositions can be suitable for oral, rectal, topical, parenteral (including subcutaneous, intramuscular, and intravenous), ocular (ophthalmic), pulmonary (nasal or buccal inhalation), or nasal administration, although the most suitable route in any given case will depend on the nature and severity of the conditions being treated and on the nature of the active ingredient. They may be conveniently presented in unit dosage form and prepared by any of the methods well-known in the art of pharmacy.

In practical use, the compounds described herein can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions as oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, hard and soft capsules and tablets, with the solid oral preparations being preferred over the liquid preparations.

Because of their ease of administration, tablets and capsules represent the most advantageous oral dosage unit form in which case solid pharmaceutical carriers are employed. If desired, tablets may be coated by standard aqueous or nonaqueous techniques. Such compositions and preparations should contain at least 0.1 percent of active compound. The percentage of active compound in these compositions may, of course, be varied and may conveniently be between about 2 percent to about 60 percent of the weight of the unit. The amount of active compound in such therapeutically useful compositions is such that an effective dosage will be obtained. The active compounds can also be administered intranasally as, for example, liquid drops or spray.

The tablets, pills, capsules, and the like may also contain a binder such as gum tragacanth, acacia, corn starch or gelatin; excipients such as dicalcium phosphate; a disintegrating agent such as corn starch, potato starch, alginic acid; a lubricant such as magnesium stearate; and a sweetening agent such as sucrose, lactose or saccharin. When a dosage unit form is a capsule, it may contain, in addition to materials of the above type, a liquid carrier such as a fatty oil.

Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor.

For ophthalmic applications, the therapeutic compound is formulated into solutions, suspensions, and ointments appropriate for use in the eye. For ophthalmic formulations generally, see Mitra (ed.), Ophthalmic Drug Delivery Systems, Marcel Dekker, Inc., New York, N.Y. (1993) and also Havener, W. H., Ocular Pharmacology, C. V. Mosby Co., St. Louis (1983). Ophthalmic pharmaceutical compositions may be adapted for topical administration to the eye in the form of solutions, suspensions, ointments, creams or as a solid insert. For a single dose, from between 0.1 ng to 5000 μg, 1 ng to 500 μg, or 10 ng to 100 μg of the aromatic-cationic peptides can be applied to the human eye.

The ophthalmic preparation may contain non-toxic auxiliary substances such as antibacterial components which are non-injurious in use, for example, thimerosal, benzalkonium chloride, methyl and propyl paraben, benzyldodecinium bromide, benzyl alcohol, or phenylethanol; buffering ingredients such as sodium chloride, sodium borate, sodium acetate, sodium citrate, or gluconate buffers; and other conventional ingredients such as sorbitan monolaurate, triethanolamine, polyoxyethylene sorbitan monopalmitylate, ethylenediamine tetraacetic acid, and the like.

The ophthalmic solution or suspension may be administered as often as necessary to maintain an acceptable level of the alpha helix mimetic β-catenin inhibitor in the eye. Administration to the mammalian eye may be about once or twice daily.

Compounds described herein may also be administered parenterally. Solutions or suspensions of these active compounds can be prepared in water suitably mixed with a surfactant or mixture of surfactants such as hydroxypropylcellulose, polysorbate 80, and mono and diglycerides of medium and long chain fatty acids. Dispersions can also be prepared in glycerol, liquid polyethylene glycols and mixtures thereof in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), suitable mixtures thereof, and vegetable oils.

The present disclosure is further illustrated by the following non-limiting examples.

### EXAMPLES

### Example 1

### PVR Study

The objective of this study was to assess the anti-fibrotic efficacy of Compound A, an alpha helix mimetic β-catenin inhibitor compound, in a rat model of proliferative vitreoretinopathy (PVR) following retinal detachment. Compound A is 4-(((6S,9S,9aS)-1-(benzylcarbamoyl)-2,9-dimethyl-4,7-dioxo-8-(quinolin-8-ylmethyl)octahydro-1H-pyrazino[2,1-c][1,2,4]triazin-6-yl)methyl)phenyl dihydrogen phosphate.

The well defined normal consequences of retinal detachment in this animal model are the hyperproliferation of retinal glial cells (primarily Muller cells), the recruitment of immune cells, and the formation of glial scars. An effective treatment would result in less glial scarring.

Retinal detachments were created by infusing a dilute solution (0.25%) of Healon into the subretinal space of the right eyes in 16 Long Evans rats. Twenty (20) mg/ml of Compound A in 5 microliters were injected intravitreally immediately after the detachment surgery in 8 animals. The other 8 animals received an intravitreal injection of the vehicle as a control. The left eyes served as naive controls. Seven days after detachment, settling of the retina occurs causing folds to form in the retina. All animals were euthanized using CO2, 7 days after retinal detachment.

Following euthanasia, the retinas were fixed in 4% paraformaldehyde for 24 hours. Three retinal regions approximately 3 mm square were sampled from within each detached retina as well as from control retinas. The retinas were embedded in agarose and vibratomed at 100 microns in thickness. Sections were immunolabeled with antibodies to intermediate filament proteins (vimentin) and proliferating cells (phosphohistone H3). A marker for immune cells (isolectin B4) and a nuclear stain (Hoescht) was also used. All 4 probes were added to the same sections (i.e. quadruple labeling).

The sections were imaged using an Olympus FV1000 confocal microscope. Digital images were aquired and used to determine 1) the number and size of subretinal glial scars 2) the number of dividing cells and their cell type e.g. of immune or glial origin 3) whether microglia were “activated” and 4) if macrophages were present.

The data were analyzed using a two-tailed T-test. Mean differences with a P values less than 0.05 were considered significant.

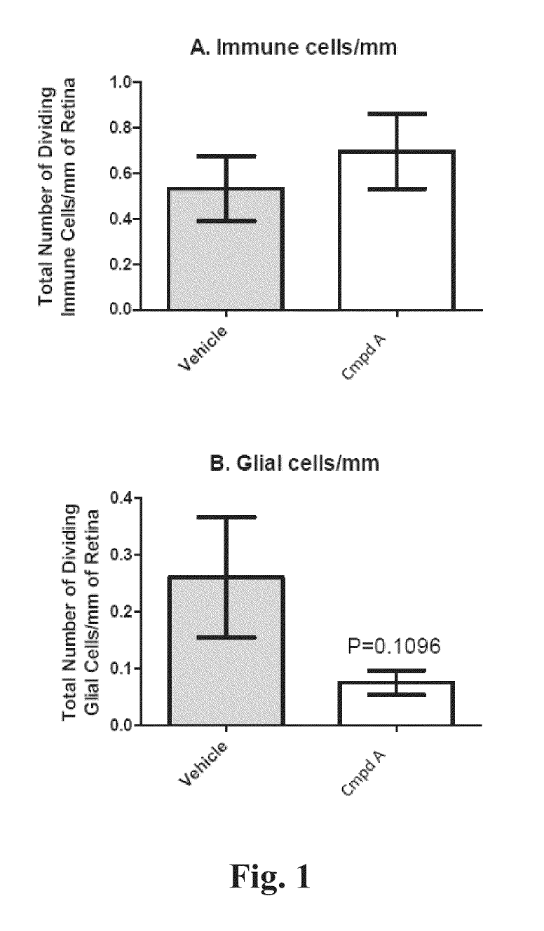

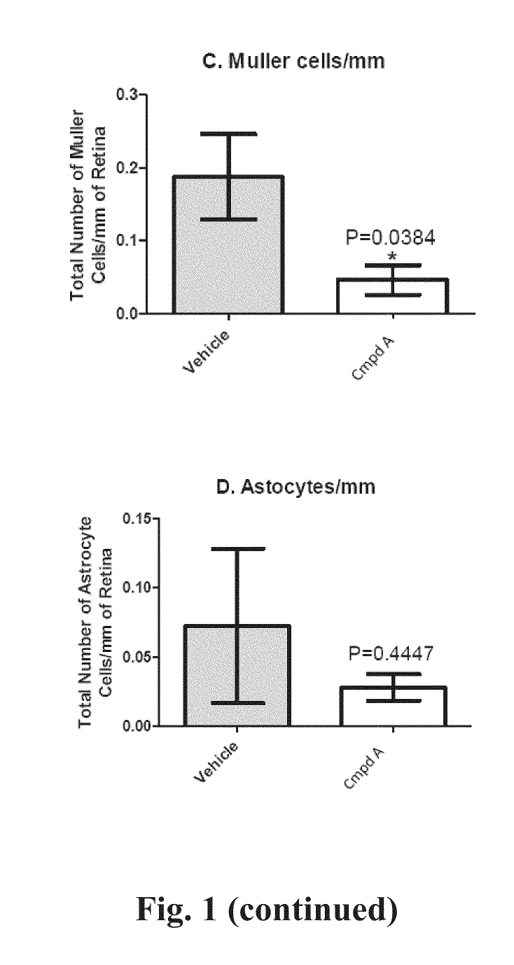

Specific histological stains were utilized in this study to determine the number of dividing immune cells present in the areas of retinal detachment as well as the number of dividing glial cells (astrocytes and Muller cells). The results from this analysis are summarized in FIGS. 1A-1D. There was an increase in dividing immune cells following retinal detachment when compared to naïve retina (data not shown). Compound A treatment did not have any effect on this immune cell response (FIG. 1A). Retinal detachment typically results in an increased number of proliferating glial cells, Muller cells, and astrocytes. This increase was largely attenuated in eyes treated with Compound A (FIGS. 1B-1D). The number of Muller cells/mm was significantly affected by Compound A treatment (FIG. 1C).

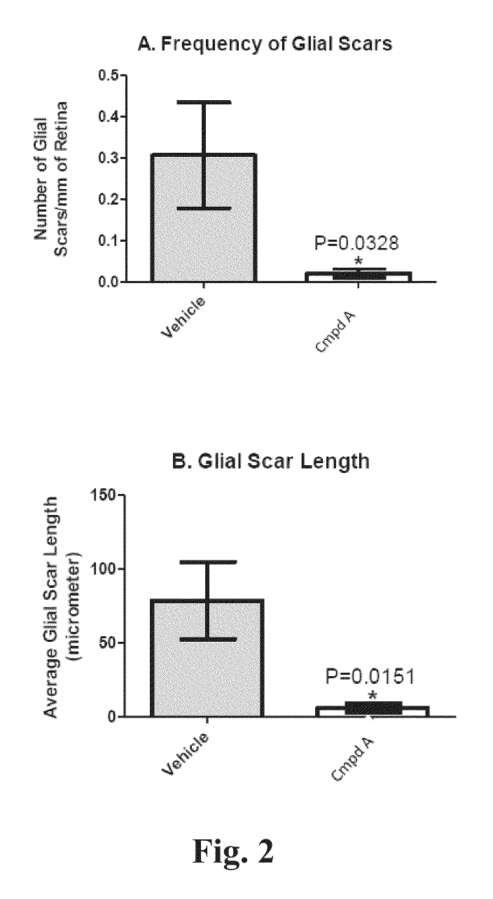

The biological significance of this reduction on proliferating Muller cells is further exhibited in Compound A's effect on glial scar formation (FIGS. 2A-2B). Scar frequency was calculated by dividing the total number of scars noted in each retina by the total area examined. The average scar size/length was calculated by dividing the total scar length in a given retina by the total number of scars counted in that retina. Compound A significantly reduced the frequency of glial scar formation (FIG. 2A) and also resulted in a significant reduction in average scar size (FIG. 2B).

Immunohistochemistry. Qualitative assessment of animals (4 saline and 4 Compound A treated) were evaluated for glial scarring, cell proliferation, and immune cell infiltrates. Three retinal regions from rats 5-8 (PVR+saline) and 13-16 (PVR+Compound A) were excised from the eye, sectioned, and labeled with antibodies (˜25 sections from each eye were surveyed).

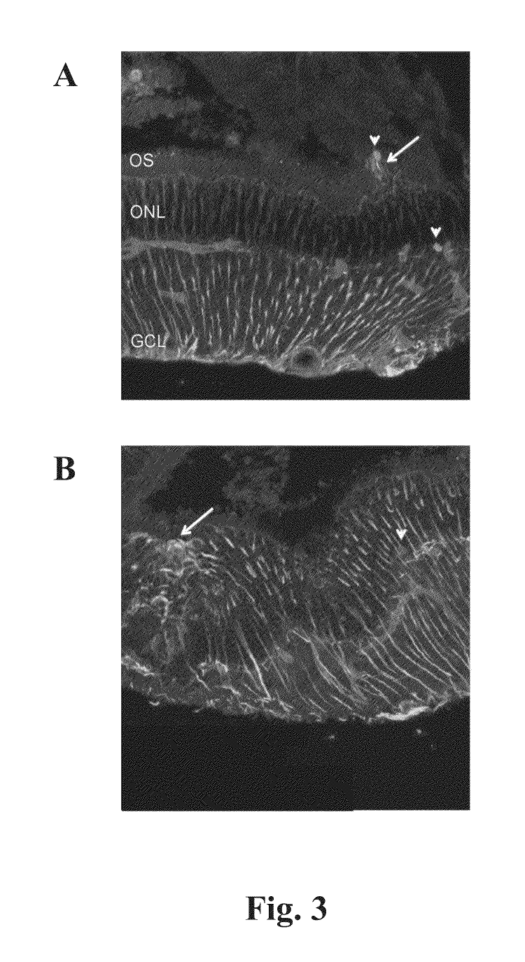

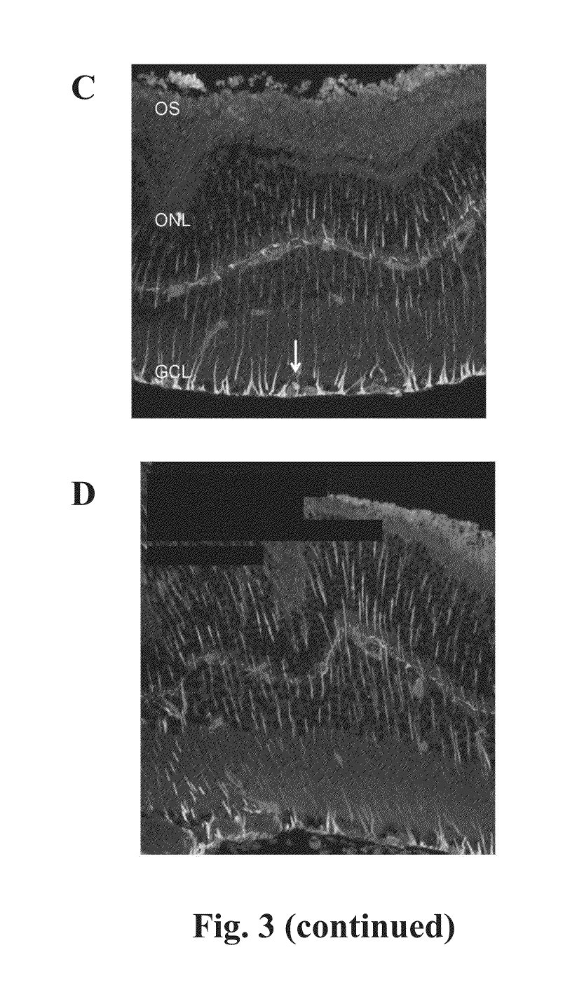

Subretinal gliosis (or scarring), defined as the presence of vimentin labeled Muller cell processes extending into the subretinal space, was observed in saline treated eyes (FIGS. 3A-3B), while no glial scarring was noted in any of the Compound A treated animals examined (FIGS. 3C-3D).

Thus, administration of Compound A treats or prevents glial scarring.

### Example 2

### CNV Study

The objective of this study was to assess the anti-angiogenic/vascular disrupting effects of Compound A, an alpha helix mimetic β-catenin inhibitor compound, and Compound C, the active metabolite of Compound A, in a rat model of laser-induced choroidal neovascularization. Compound A is 4-(((6S,9S,9aS)-1-(benzylcarbamoyl)-2,9-dimethyl-4,7-dioxo-8-(quinolin-8-ylmethyl)octahydro-1H-pyrazino[2,1-c][1,2,4]triazin-6-yl)methyl)phenyl dihydrogen phosphate. Compound C is (6S,9S,9aS)-N-benzyl-6-(4-hydroxybenzyl)-2,9-dimethyl-4,7-dioxo-8-(quinolin-8-ylmethyl)octahydro-1H-pyrazino[2,1-c][1,2,4]triazine-1-carboxamide.

For compound A, an 80 mg/ml solution of 4-(((6S,9S,9aS)-1-(benzylcarbamoyl)-2,9-dimethyl-4,7-dioxo-8-(quinolin-8-ylmethyl)octahydro-1H-pyrazino[2,1-c][1,2,4]triazin-6-yl)methyl)phenyl dihydrogen phosphate was prepared in sterile PBS. This solution was further diluted 1:4 to make a 20 mg/ml solution. The 20 mg/ml solution was diluted 1:4 to make a 5 mg/ml solution.

For compound C, a solution containing 0.5% NaCMC and 0.5% Polysorbate 80 (Tween 80) was prepared in USP grade water. 20 mg of (6S,9S,9aS)-N-benzyl-6-(4-hydroxybenzyl)-2,9-dimethyl-4,7-dioxo-8-(quinolin-8-ylmethyl)octahydro-1H-pyrazino[2,1-c][1,2,4]triazine-1-carboxamide was dissolved in 1 ml (using displacement pipette) of USP grade PEG400 in a glass vial. Slight heating and sonication/vortexing were performed if necessary. The glass vial containing the 20 mg/ml compound C solution was placed on a magnetic stir plate and an equal volume (1 ml) of the CMC/Tween 80 solution was added to the compound C/PEG400 solution (slowly in drop-wise fashion during continuous mixing with a stir bar). The resultant formulation was a clear solution containing 10 mg/ml compound C, 50% PEG400, 0.25% NaCMC, and 0.25% Tween 80.

Laser application to produce CNV lesions. Animals were dilated with 1% Cyclogyl solution and protected from light. Following observable dilation, the animals were sedated with ketamine/xylazine. The fundus of sedated animals was observed and recorded using a Micron III small animal funduscope (Phoenix Research). Laser treatments were performed using a thermal laser which is connected through the Micron III custom laser attachment. A total of 3 lesions per eye were placed using a wavelength of 520 nm.

Fundus images were recorded to confirm that the laser had successfully produced a bubble through the Bruch's membrane. It was expected that 5-10% of all laser spots would not develop any quantifiable CNV.

Intravitreal injections. Animals were anesthetized with ketamine/xylazine and the test compound was then injected in a volume of 5 μl into the vitreous through the pars plana using a Hamilton syringe and a 32 gauge needle. Following injection, the animals received an equal amount of topical antibiotic ointment on both eyes. Any eyes displaying signs of hemorrhage following laser application or intravitreal injection were excluded from analysis.

Fluorescein angiography. Animals were anesthetized with ketamine/xylazine and then received an IP injection of 10% Fluorescein Sodium at 1 μl/gram of body weight. Fundus images were then captured as 8-bitt TIFF files using the Micron III and exciter/barrier filters for a target wavelength of 488 nm. Standard color fundus photos were also captured for each eye.

Imaging and lesion quantification. All TIFF images were quantified using computerized image-analysis software (ImageJ, NIH, USA). Lesions were then individually traced free-hand in order to quantify the area in pixels and the color fundus photos were used as a reference for lesion location. Areas of avascularization in the center of lesions were excluded from area calculations. In the case of a hemorrhage or two lesions overlapping these lesions were excluded from analysis.

Statistical Analyses. Statistical Analyses were performed with Graphpad Prism software (version 5) using one-way analysis of variance (ANOVA) with a Tukey's post-hoc test for significance. Only changes with a p-value<0.05 were deemed statistically significant.

Animals. Female Brown Norway rats, 8 weeks-old at time of Laser-treatment.

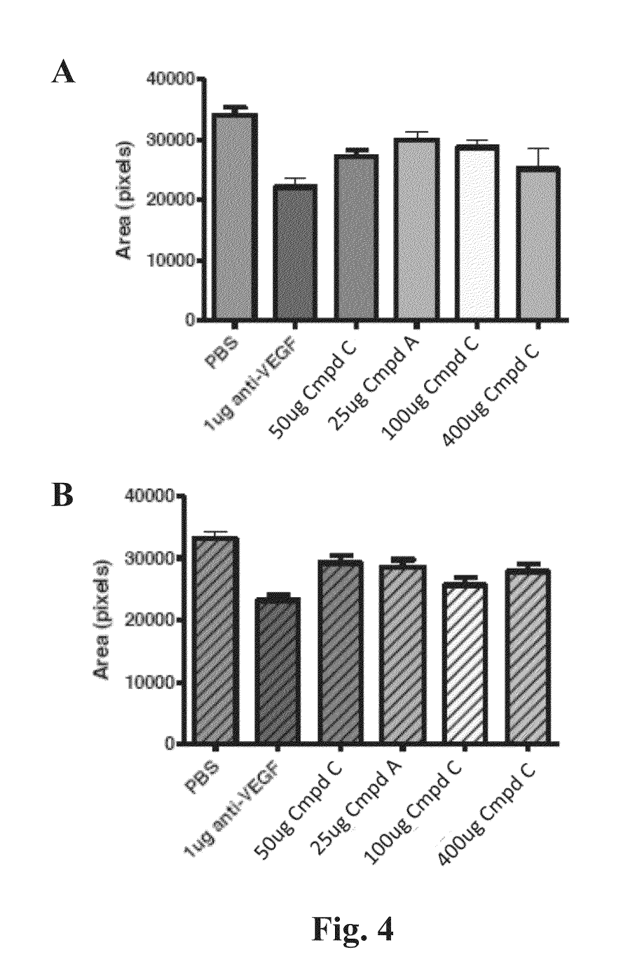

Results. The effect of two bilateral intravitreal administrations (on Days 3 and 10) of vehicle (PBS), anti-VEGF Ab (positive control), Compound A (at three different doses; 25 μg, 100 μg and 400 μg), or Compound C (50 μg) was evaluated in a rat model of laser-induced choroidal neovascularization (CNV). On Days 15 and 22 (two-and three-weeks post laser treatment) fundus imaging and fluorescein angiography were performed to quantify the size (area) of the CNV lesions in these rats. Average lesion size was smaller for all treatments at Day 15 (compared to vehicle controls), and was statistically significant for treatment groups administered the anti-VEGF antibody (positive control; p<0.001), 50 μg Compound C (p<0.05), or 400 μg Compound A (p<0.01) (FIG. 4A). At Day 22, only anti-VEGF (p<0.001) and 100 μg Compound A (p<0.01) demonstrated significance (FIG. 4B).

Both tested formulations (Compound A and Compound C) demonstrated efficacy in terms of anti-angiogenic or vascular disruption activity in a rat model of CNV. For Compound A there was a dose effect as only the two higher tested doses (100 μg or 400 μg) demonstrated statistical significance. Compound C (50 μg) also had a significant impact on the size of the lesions.

Thus, Compound A and Compound C are effective for treating and preventing neovascularization.

1. An alpha helix mimetic β-catenin inhibitor compound for the treatment of one or more ophthalmic conditions, having the following formula (I): 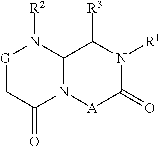
wherein:
A is —CHR⁷—,
wherein R⁷ is hydrogen, optionally substituted alkyl, optionally substituted alkenyl, optionally substituted alkynyl, optionally substituted arylalkyl, optionally substituted heteroarylalkyl, optionally substituted cycloalkylalkyl, optionally substituted heterocycloalkylalkyl, optionally substituted aryl, optionally substituted heteroaryl, optionally substituted cycloalkyl or optionally substituted heterocycloalkyl;
G is —NH—, —NR⁶—, —O—, —CHR⁶— or —C(R⁶)₂—,
wherein R⁶ is independently selected from optionally substituted alkyl, optionally substituted alkenyl and optionally substituted alkynyl;
R¹ is optionally substituted arylalkyl, optionally substituted heteroarylalkyl, optionally substituted cycloalkylalkyl or optionally substituted heterocycloalkylalkyl;
R² is —W²¹—W²²—Rb—R²⁰,
wherein W²¹ is —(CO)— or —(SO₂)—; W²² is bond, —O—, —NH— or optionally substituted lower alkylene; Rb is bond or optionally substituted lower alkylene; and R²⁰ is optionally substituted alkyl, optionally substituted alkenyl, optionally substituted alkynyl, optionally substituted aryl, optionally substituted heteroaryl, optionally substituted cycloalkyl or optionally substituted heterocycloalkyl; and
R³ is optionally substituted alkyl, optionally substituted alkenyl or optionally substituted alkynyl;or a pharmaceutically acceptable salt thereof.

2. The compound of claim 1, selected from:
(6S,9S)—N-benzyl-6-(4-hydroxybenzyl)-2,9-dimethyl-8-(naphthalen-1-ylmethyl)-4,7-dioxooctahydro-1H-pyrazino[2,1-c][1,2,4]triazine-1-carboxamide,
(6S,9S)-2-allyl-N-benzyl-6-(4-hydroxybenzyl)-9-methyl-8-(naphthalen-1-ylmethyl)-4,7-dioxooctahydro-1H-pyrazino[2,1-c][1,2,4]triazine-1-carboxamide,
(6S,9S)—N-benzyl-6-(4-hydroxybenzyl)-9-methyl-8-(naphthalen-1-ylmethyl)-4,7-dioxohexahydropyrazino[2,1-c][1,2,4]oxadiazine-1(6H)-carboxamide,
(6S,9S)-8-((2-aminobenzo[d]thiazol-4-yl)methyl)-N-benzyl-6-(4-hydroxybenzyl)-2,9-dimethyl-4,7-dioxooctahydro-1H-pyrazino[2,1-c][1,2,4]triazine-1-carboxamide,
(6S,9S)—N-benzyl-6-(4-hydroxybenzyl)-2,9-dimethyl-4,7-dioxo-8-(quinolin-8-ylmethyl)octahydro-1H-pyrazino[2,1-c][1,2,4]triazine-1-carboxamide,
(6S,9S)-2-allyl-N-benzyl-6-(4-hydroxybenzyl)-9-methyl-4,7-dioxo-8-(quinolin-8-ylmethyl)octahydro-1H-pyrazino[2,1-c][1,2,4]triazine-1-carboxamide,
4-(((6S,9S)-1-(benzylcarbamoyl)-2,9-dimethyl-4,7-dioxo-8-(quinolin-8-ylmethyl)octahydro-1H-pyrazino[2,1-c][1,2,4]triazin-6-yl)methyl)phenyl dihydrogen phosphate,
4-(((6S,9S)-1-(benzylcarbamoyl)-2,9-dimethyl-8-(naphthalen-1-ylmethyl)-4,7-dioxooctahydro-1H-pyrazino[2,1-c][1,2,4]triazin-6-yl)methyl)phenyl dihydrogen phosphate,
sodium 4-(((6S,9S)-1-(benzylcarbamoyl)-2,9-dimethyl-4,7-dioxo-8-(quinolin-8-ylmethyl)octahydro-1H-pyrazino[2,1-c][1,2,4]triazin-6-yl)methyl)phenyl phosphate,
sodium 4-(((6S,9S)-1-(benzylcarbamoyl)-2,9-dimethyl-4,7-dioxo-8-(naphthalen-8-ylmethyl)octahydro-1H-pyrazino[2,1-c][1,2,4]triazin-6-yl)methyl)phenyl phosphate,
(6S,9S)-2-allyl-6-(4-hydroxybenzyl)-9-methyl-4,7-dioxo-N—((R)-1-phenylethyl)-8-(quinolin-8-ylmethyl)octahydro-1H-pyrazino[2,1-c][1,2,4]triazine-1-carboxamide,
(6S,9S)-2-allyl-6-(4-hydroxybenzyl)-9-methyl-4,7-dioxo-N—((S)-1-phenylethyl)-8-(quinolin-8-ylmethyl)octahydro-1H-pyrazino[2,1-c][1,2,4]triazine-1-carboxamide,
(6S,9S)—N-benzyl-6-(4-hydroxy-2,6-dimethylbenzyl)-2,9-dimethyl-4,7-dioxo-8-(quinolin-8-ylmethyl)octahydro-1H-pyrazino[2,1-c][1,2,4]triazine-1-carboxamide,
(6S,9S)-8-(benzo[b]thiophen-3-ylmethyl)-N-benzyl-6-(4-hydroxybenzyl)-2,9-dimethyl-4,7-dioxooctahydro-1H-pyrazino[2,1-c][1,2,4]triazine-1-carboxamide,
(6S,9S)-8-(benzo[c][1,2,5]thiadiazol-4-ylmethyl)-N-benzyl-6-(4-hydroxybenzyl)-2,9-dimethyl-4,7-dioxooctahydro-1H-pyrazino[2,1-c][1,2,4]triazine-1-carboxamide,
(6S,9S)—N-benzyl-6-(4-hydroxybenzyl)-8-(isoquinolin-5-ylmethyl)-2,9-dimethyl-4,7-dioxooctahydro-1H-pyrazino[2,1-c][1,2,4]triazine-1-carboxamide,
(6S,9S)—N-benzyl-8((5-chlorothieno[3,2-b]pyridin-3-yl)methyl)-6-(4-hydroxybenzyl)-2,9-dimethyl-4,7-dioxooctahydro-1H-pyrazino[2,1-c][1,2,4]triazine-1-carboxamide,
(6S,9S)—N-benzyl-6-(4-hydroxybenzyl)-2,9-dimethyl-4,7-dioxo-8-(quinoxalin-5-ylmethyl)octahydro-1H-pyrazino[2,1-c][1,2,4]triazine-1-carboxamide, and
(6S,9S)-6-(4-hydroxybenzyl)-2,9-dimethyl-4,7-dioxo-8-(quinolin-8-ylmethyl)-N-(thiophen-2-ylmethyl)octahydro-1H-pyrazino[2,1-c][1,2,4]triazine-1-carboxamide.

3. The compound of claim 1, selected from:
4-(((6S,9S,9aS)-1-(benzylcarbamoyl)-2,9-dimethyl-4,7-dioxo-8-(quinolin-8-ylmethyl) octahydro-1H-pyrazino[2,1-c][1,2,4]triazin-6-yl)methyl)phenyl dihydrogen phosphate, and
(6S,9S,9aS)-N-benzyl-6-(4-hydroxybenzyl)-2,9-dimethyl-4,7-dioxo-8-(quinolin-8-ylmethyl)octahydro-1H-pyrazino[2,1-c][1,2,4]triazine-1-carboxamide.

4. A pharmaceutical composition comprising the compound of claim 1.

5. The compound of claim 1 wherein the one or more ophthalmic conditions is selected from macular degeneration, age-related macular degeneration, glaucoma, cataracts, retinitis pigmentosa, choroidal neovascularization, retinal degeneration, and oxygen-induced retinopathy.

6. A method of treatment for an ophthalmic condition, comprising administering an effective amount of the compound of claim 1.

7. The method of claim 6, wherein the condition is glaucoma.

8. The method of claim 6, wherein the condition is macular degeneration.

9. The method of claim 8, wherein the condition is age-related macular degeneration.
